(19) United States
(12) Patent Application Publication
(10) Pub. No.: US 2025/0352288 A1
(43) Pub. Date: Nov. 20, 2025

(54) A CONTROL METHOD OF A ROBOTIC SYSTEM FOR MEDICAL OR SURGICAL TELEOPERATION AND RELATED ROBOTIC SYSTEM

(71) Applicant: Medical Microinstruments, Inc., Wilmington, DE (US)

(72) Inventors: Emanuele Ruffaldi, Pisa (PI) (IT); Massimiliano Simi, Pisa (PI) (IT); Antonio Di Guardo, Pisa (PI) (IT)

(21) Appl. No.: 18/871,342

(22) PCT Filed: May 30, 2023
(86) PCT No.: PCT/IB2023/055511

§ 371 Date: Dec. 3, 2024

(30) Foreign Application Priority Data
Jun. 3, 2022 (IT) 102022000011789

(51) Int. Cl. A61B 34/35; A61B 17/00; A61B 34/00; A61B 34/20; A61B 90/00

(52) U.S. Cl. CPC A61B 34/35; A61B 34/20; A61B 34/25; A61B 2017/00207; A61B 2034/2055; A61B 2034/252; A61B 90/37

(57) ABSTRACT

A control method usable in any robotic system for medical or surgical teleoperation having at least one master device, at least one slave device configured to be moved by an actuator and to be controlled by the master device, a central microprocessor unit configured to control the slave device and a display configured to visualize in its central portion a surgical site in which the slave device operates, comprises the operations of; determining with the central unit the spatial orientations of the master and slave devices; when the central unit receives an help-alignment request signal, generating a graphical user interface on the display comprising at least one help-alignment graphic element visually representing a relative spatial orientation of the master device with respect to the slave device; modifying at least one graphical aspect of the help-alignment graphic element in a manner corresponding to the relative spatial orientation of the master device with respect to the slave device.

### TECHNICAL FIELD

The present invention relates to a control method of a robotic system for medical or surgical teleoperation.

In particular, the present invention relates to a control method for starting a medical or surgical teleoperation.

The present invention also relates to a related robotic system for medical or surgical teleoperation.

### BACKGROUND

In a master-slave teleoperation where both the master and slave devices have rotational degrees of freedom, the operator typically provides a command to enable the teleoperation, such as pressing a foot pedal and/or pressing a button available on the master device body. As is known, during master-slave teleoperation the master device position is mapped in the slave workspace as target position.

In preparing a teleoperation there is a common problem to the various master-slave solutions which is the respective alignment of the orientation, as it is highly desirable that during a phase of medical or surgical teleoperation the master and slave are aligned, i.e., for example, they are perceived as aligned by the operator who performs the surgical gesture.

This problem is particularly felt when a non-actuated or “flying” master device is used, i.e. without force feedback, for unilateral teleoperation, such as for example in the case in which the master device is mechanically not linked to the master operating console.

In fact, in an ideal case, a teleoperation should only start when master and slave are perfectly aligned in orientation or when they are misaligned by a very small angle, in order to preserve and guarantee coherence between the direction of motion and the orientation of the user who controls the master device and motion direction and orientation of the slave device.

Conversely, it is generally considered undesirable to teleoperate with master and slaves misaligned beyond a certain misalignment threshold (e.g. 15 degrees solid angle), because this results in counterintuitive movement of the slave device with consequent potential risks arising.

For these reasons, an alignment phase is typically provided which is preparatory to the fully slaved teleoperation phase, which has the purpose of minimizing the misalignment between the master device and the slave device before enabling entry into teleoperation.

During an alignment step preparatory to teleoperation a further usability requirement arises, whereby it is desirable to be able to enter teleoperation in any case even if a residual master-slave misalignment is present.

A solution proposed in document PCT/IB2022/051226 in the name of the same Applicant provides, in the alignment phase preparatory to the teleoperation, an alignment sub-phase with movement, in which the slave device is enabled to move to align itself with the master device, when the misalignment is lower than a certain predeterminable threshold and if one or more further checks are satisfied. In this case, the need is also felt, during the alignment phase, to avoid or at least reduce to a minimum the movement of the slave device for alignment to the master device. This document also discloses an alignment step in which the slave device follows the master device only in the orientation, avoiding translation.

Solutions have also been proposed in which the teleoperation is allowed even in the presence of a master-slave misalignment, and this misalignment is gradually recovered during the teleoperation.

From the constructive point of view, master consoles with a mechanically constrained and motorized appendix which act as a “master controller” device are known in the master-slave robotic systems for medical or surgical teleoperation. In such robotic systems, typically, the motors of the “master controller” appendage impose on the master device a condition of alignment to the current orientation of the slave device in the slave workspace, limiting the movement in orientation of the master device; in other words, these systems prevent misalignment by blocking the master device linked to the console.

Also known are master devices for medical or surgical teleoperation mechanically linked to the console by means of a universal support and stabilization system equipped with a gyroscope (“gimbal”).

Master devices have also been proposed for medical or surgical teleoperation mechanically linked directly to one or more slave robotic arms to move said one or more slave robotic arms, as for example shown in WO-2016-030767.

Solutions have recently emerged with master devices for medical or surgical teleoperation that are not mechanically linked to the console of the robotic system, i.e. unbound, or “ungrounded” or “groundless” master devices, i.e. of the type as shown for example in documents WO-2019-220407, WO-2019-220408, WO-2019-220409, WO-2021-161158, WO-2021-161185 and WO-2021-161177 in the name of the same Applicant, as well as of the type shown for example in documents U.S. Pat. No. 8,521,331, US-2020-0360097 and WO-2016-137527.

Another category of master devices is that of the non-actuated or “flying” type, i.e. without feedback systems coming from the slave device which could physically limit its maneuverability. This category can include both master devices of the mechanically non-constrained type mentioned above and master devices of the type constrained to the operating console, for example where a gimbal for support and stabilization (“gimbal”) is provided.

Known robotic systems generally comprise at least one master device, which can be gripped, adapted to be moved by an operator, at least one slave device configured to be moved by an actuator and to be controlled by the master device, a central microprocessor unit configured to control said slave device, and a display.

In known robotic systems, the mechanically unconstrained master device is tracked within a magnetic field and/or by an optical tracking system.

The robot system display can be configured to show a surgical site where the slave device is operating.

Once the alignment condition has been reached, the robotic system can enable entry into master-slave teleoperation, typically following the pressure of a control pedal.

In all such robotic systems, both those with non-actuated or flying master devices, and those with non-linked master devices but also in some cases with master devices linked to the operating console, it is important before starting a master-slave teleoperation to obtain the alignment between the master device and the slave device, to have correctly and coherently associated master-slave references, so that the surgical gesture of the operator is performed, by the slave surgical instrument, in a manner perceived as corresponding.

For example, prior document EP-3305236 shows a robotic system solution for endoscopic surgery in which information about the orientation of parts of the articulated end effector of the slave surgical instrument is shown on a display together with information about the orientation of rotational joints of the support addendum to the master control device.

For example, document US-2016-0235489 shows a solution for tracking the orientation of surgeon hands to control a slave robotic surgical instrument.

For example, the earlier document WO-2016-137527 discloses an example of master-slave misalignment display in a robotic system comprising a master device of the unconstrained type and freely movable in space (UID).

For example, the prior document WO-2019-103954 shows a master slave surgical teleoperation system in which an orientation of the master control device with respect to a reference thereof corresponds to an orientation of the slave surgical instrument with respect to the image acquisition device.

### SUMMARY

An object of the present disclosure is to provide a control method which overcomes the limitations of known methods in the master-slave alignment step prior to teleoperation.

A further object of the present disclosure is to propose a solution capable of making the alignment step simpler and more intuitive before starting a master-slave teleoperation step.

Thanks to the proposed solutions, it is possible to provide visual aid-alignment elements able to guide the surgeon in an alignment phase to allow starting a teleoperation phase in such a way as to combine usability needs and at the same time reducing risks to a minimum.

In particular, thanks to the proposed solutions, it is possible to reduce the time necessary for the alignment step and therefore the time necessary for the preparation of a completely slaved teleoperation step is reduced.

Thanks to the solutions proposed, and particularly in those robotic systems in which an alignment phase is provided comprising an alignment sub-phase with movement, in which the slave device is enabled to move to align itself with the orientation of the master device, reducing or resetting in this way the master-slave misalignment allows to minimize—even to eliminate—the need to move the slave device during the alignment phase and consequently the path of the slave during its alignment to the master device during said alignment phase with movement.

A further object is that the invention can be used in any robotic system for medical or surgical teleoperation having at least one master device, at least one slave device configured to be moved by an actuator and to be controlled by the master device, a central unit microprocessor configured to control the slave device and a display configured to view these “HELPER”.

The display can be configured to also visualize, for example simultaneously, a surgical site in which the slave device operates.

These and other aims are achieved at least in part with the method defined in claim 1.

According to the method of this disclosure, when the central unit receives an help-alignment request signal, a graphical user interface is generated on the display comprising at least one help-alignment graphic element displayed in a portion of the display.

In one aspect, the at least one help-alignment graphic element visually represents information about a relative spatial orientation of the master device with respect to the slave device.

According to one aspect, the at least one help-alignment graphic element represents instructions for allowing said operator to achieve an alignment condition between the master device and the slave device.

According to one embodiment, the master device is of the non-actuated type.

According to one embodiment, the master device is of the type not mechanically linked to the console, i.e. it is of the “flying” type within the master workspace.

According to one embodiment, the master device has N-fold symmetry along an axis, i.e. it is equal to itself for rotations equal to one part out of N of the round angle.

According to an embodiment, the step of generating on the display a graphical user interface comprising at least one help-alignment graphic element is activated during a teleoperation preparation step.

According to one embodiment, the method is performed during an alignment step, preparatory to teleoperation.

According to an embodiment, the step of generating on the display a graphical user interface comprising at least one help-alignment graphic element is activated when the master device has been detected inside and/or outside a predefined volume.

According to an embodiment, the step of generating on the display a graphical user interface comprising at least one help-alignment graphic element is activated when the master device is subjected to a predetermined hand gesture, in a predefined time interval. The hand gesture is preferably detected by detection of the master device (for example by optical and/or electromagnetic tracking).

According to an embodiment, the step of generating on the display a graphical user interface comprising at least one help-alignment graphic element is activated when the master device has been detected within a predefined volume and at the same the opening/closing command of the master device has been activated, and preferably of two master devices (right and left) at the same time. For example, the opening and closing command may have been activated repeatedly (“double-tap”).

According to one embodiment, the step of generating a graphical user interface on the display comprising at least one help-alignment graphic element is activated when the master device has been detected within a predefined volume and contextually has been turned over (“top-down”, to indicate that the surgeon's gesture is voluntarily aimed at starting the alignment phase), and, preferably, when two master devices (right and left) are turned upside down at the same time.

According to one embodiment, the orientation of the slave device is calculated from the internal measurements of the sensors or actuators of the robotic system.

According to one embodiment, the orientation of the slave device is calculated starting from an algorithm which analyzes the image of the slave acquired by a vision system.

According to one embodiment, the orientation of the slave device is calculated on the basis of the combination of the internal information of the sensors or actuators of the robotic system with those of the vision system.

Also described is a robotic system configured to implement the described methods thanks to a suitable computer program loaded into a central microprocessor unit.

Further embodiments are defined in the attached claims.

### BRIEF DESCRIPTION OF THE DRAWINGS

FIG. 1 schematically illustrates an embodiment of a robotic system for medical and/or surgical teleoperation;

FIG. 2A illustrates an example of interaction between a master device and a slave device, wherein the slave device is adapted to be enabled to move to align with the master device;

FIG. 2B schematically illustrates a slave device having an articulated terminal, in which a control point is defined which during an alignment step can only perform pure rotational movements in the slave working space;

FIG. 3 is an exemplary diagram of how to display two help-alignment graphic elements according to the present disclosure to represent a relative orientation of the respective slave device with respect to the corresponding master device;

FIG. 4 is a diagram of a procedure for updating the appearance of an help-alignment graphic element according to the present disclosure based on the relative orientation between the master device and the corresponding slave device;

FIG. 5 shows how to display two help-alignment graphic elements according to the present disclosure in the form of an annulus;

FIGS. 6A to 6F show examples of circular crown-shaped help-alignment graphic elements for representing a relative orientation of the slave device with respect to the corresponding master device;

FIG. 7 shows an example of two graphic aid-alignment elements, respectively right and left, in the shape of three-dimensional cones;

FIG. 8 shows an example of a detail of an help-alignment graphic element depicted in FIG. 7, in which two three-dimensional cones respectively represent the orientation of the master device and the relative orientation of the slave device;

FIG. 9 shows an example of two aid-alignment graphic elements, respectively right and left, in the shape of three-dimensional cones and two-color colouring;

FIG. 10 shows an example of a detail of an help-alignment graphic element illustrated in FIG. 9, wherein two three-dimensional cones respectively represent the orientation of the master device and the relative orientation of the slave device;

FIG. 11 shows an example of two help-alignment graphic elements according to the present disclosure depicting the orientation of the master devices and respective slave devices superimposed in a composite image;

FIG. 12 shows an example of a detail of an help-alignment graphic element illustrated in FIG. 11, in which the orientation of a slave device is illustrated in a synthetic image partially superimposed on an image of a respective master device;

FIGS. 13A, 13B and 13C show other examples of an help-alignment graphic element according to the present disclosure for representing a relative orientation of the slave device with respect to the corresponding master device;

FIGS. 14A, 14B and 14C show other examples of an help-alignment graphic element according to the present disclosure for providing instructions to the operator in order to achieve alignment between the master device and the slave device;

FIGS. 15 to 18 show state diagrams illustrating different ways to start a teleoperation after opening a graphical user interface on the display of a robotic system according to the method of the invention;

FIG. 19 is a graph showing the trend over time of the master-slave misalignment, according to a possible operating method,

FIG. 20 is a graph showing the trend over time of the master-slave misalignment, according to a possible operating method;

FIGS. 21 to 24 schematically show a step of generating the help-alignment request signal, according to some possible operating modes.

### DETAILED DESCRIPTION OF ILLUSTRATIVE EMBODIMENTS

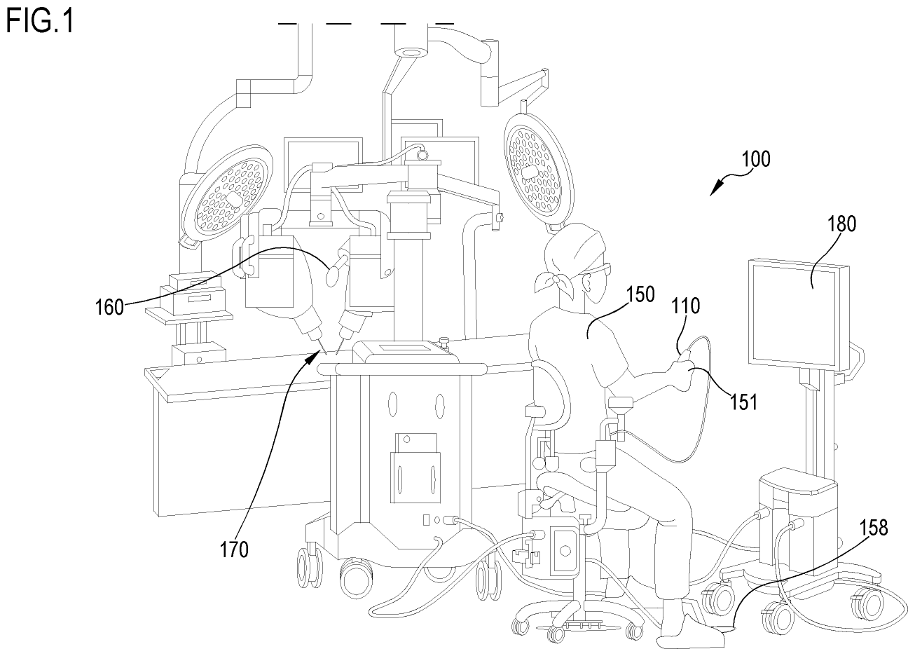

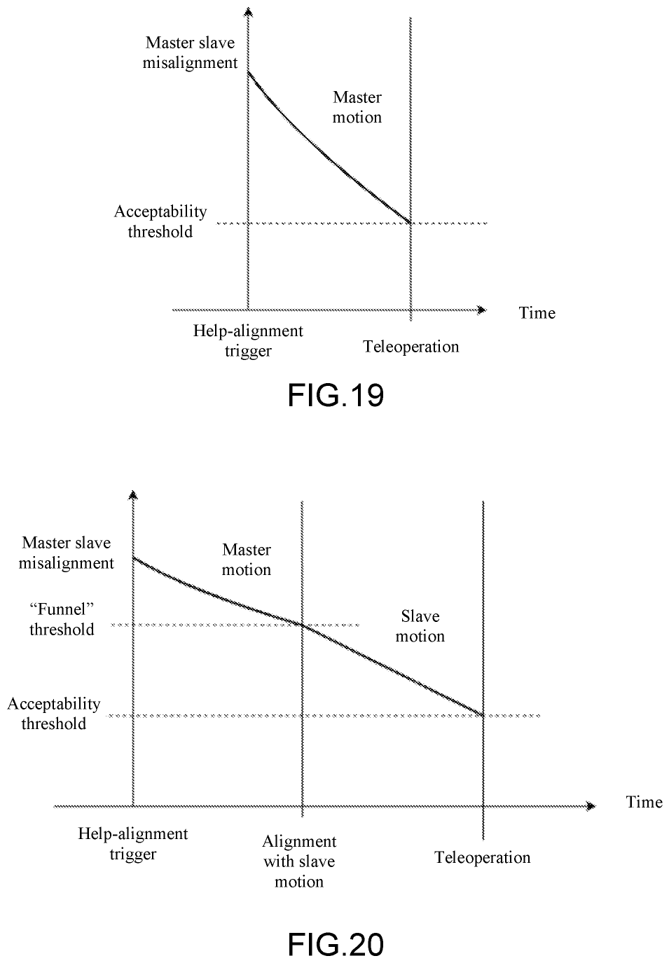

In the ensuing description, a control method of a robotic system 100 for medical or surgical teleoperation will be described with reference to FIGS. 1 to 24. In general, a robotic system 100 in which a control method of this disclosure can be implemented is of the type schematically illustrated in FIG. 1 and includes at least:
a master device 110, which can be held, adapted to be handled by an operator 150,
a slave device 170, which can have at least one surgical instrument 170 and which is configured to be controlled by the master device 110 and to be moved by an actuator so as to track the movements of the master device,
a central microprocessor unit configured to control the slave device and to move it via one or more dedicated actuators, and a display 180.

The display 180 can be configured to display a surgical site where the slave device 170 is operating.

Robotic system 100 may include two slave devices (i.e., two slave surgical instruments) and two master devices for controlling them.

In accordance with an embodiment, said display 180 is configured to display the at least one aid-alignment graphic element 190, avoiding displaying the surgical site.

In accordance with one embodiment, at least two displays are provided, wherein one display is configured to display the surgical site and the other display is configured to display the at least one help-alignment graphic element 190. For example, the display which displays the at least one help-alignment graphic element 190 may be placed in proximity to the surgeon, for example may be worn by the surgeon as for example a glove, a mask, a pair of glasses, a bracelet, a band, a belt and/or similar.

In the example illustrated in FIG. 1, reference is made to the case of particular interest in which the master device, held by the surgeon, is mechanically not constrained to the console. This type of master devices are generally not actuated, i.e. they do not have feedback systems (joints) coming from the slave device which could physically limit their manoeuvrability, for example in the event of a master-slave misalignment detected above a certain threshold. The method of this disclosure will be illustrated with reference to this situation of particular interest, typical in the field of telemedicine, but it is understood that what will be said also applies in the case in which the master device is of the non-actuated type, whether linked or non-mechanically linked to the console, as well as where the master device is mechanically linked to the console, as well as where the master device is mechanically linked to the slave device such as a slave robotic arm.

In particular, in the case of unconstrained or “flying” master devices 110, the operator feels the need to check whether the master device, that the user is holding, is aligned with the slave device that he sees represented on the display 180 of the system robotic, since the orientation of the master device 110 with respect to the user's view may not correspond to the orientation of the surgical instrument 170 of the slave device with respect to the view of the vision system (for example a video camera 160) which records it. This need is particularly felt in a phase of preparation for medical or surgical teleoperation, so that upon entry into master-slave teleoperation, the misalignment between the master device 110 controlled by the operator and the slave device 170 as shown on the display 180 is minimal or nil.

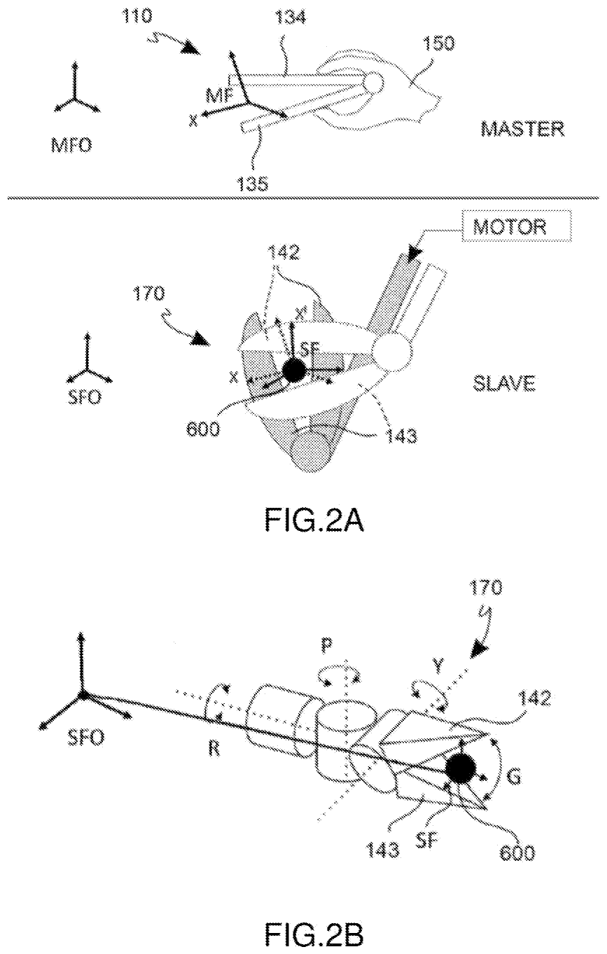

An example of a master device and a slave device is shown in FIG. 2A, together with the coordinate systems MF, SF which identify their respective orientation in space with respect to a reference MFO, SFO. In the example of FIG. 2A, the body of the master device 110 comprises two rigid parts 134, 135 integral with respective sensors or markers (not shown) constrained in a rotational joint 136 like a gripper to rotate around a common axis while the master device is manipulated by an operator 150.

In the example illustrated in FIG. 2A, a possible alignment step preparatory to the teleoperation is schematically shown in which the slave device 170 is enabled to move in the slave workspace (identified in the figure by the reference SFO) to align itself with the master device 110 (in the illustrated example it is shown with shading and in solid line the starting pose of the slave surgical instrument 170 and without shading in dashed line the target pose of the slave surgical instrument 170 which is aligned with the pose of the master device 110.

In the example illustrated in FIG. 2B, a possible alignment step is shown, in which the slave device 170 follows the master device 110 only in orientation. In other words, the control point 600 of the slave surgical instrument 170 can only perform pure rotational movements to align itself with the pose of the master device 110, independently of the positioning kinematic chain of the control point 600.

To establish the position of the master device and the slave device as well as their mutual misalignment, a control point 600 which locates the slave device can conveniently be identified. In the example of FIG. 2A, point 600 can be identified as the control point, which can belong to the slave surgical instrument and/or a virtual point rigidly associated with the slave surgical instrument. The surgical instrument of the slave device 170 may comprise a plurality of rotational joints P, Y, G, for example pitch joints P and yaw joints Y as well as opening/closing joints G, and/or the surgical instrument of the slave device can comprise a plurality of stacked vertebrae (“snake”), while the master device 110 preferably does not comprise a corresponding number of rotational joints, so thanks to the provision of the control point 600 which can be identifiable of at least six degrees of freedom of the slave device (three translations and three rotations, and if necessary the opening/closing degree of freedom G) it is possible to establish a correlation between the pose of the master device and the pose of the slave device.

The relative orientation (misalignment) between the master device 110 and the slave device 170 is generally understood as the relative rotation in the space of rotations SO(3). This relative rotation can be mathematically expressed in different ways, for example Euler angles, axis angle, or twist-swing: these representation modes can be associated with different visual solutions to help the operator in alignment. The representation with Euler angles expresses the relative rotation according to three rotations relative to orthogonal axes, the angle axis representation expresses a direction of rotation and an overall angle along the geodesic. The twist-swing representation is constructed by examining two principal directions in the slave and in the master: the swing angle is that which carries one principal direction over the other, while the twist angle is the relative angle of rotation along said principal direction.

In the robotic system 100, a vision system 160 can be provided which shoots the physical, real slave device 170 in real time, and the acquired image is displayed on said display 182. In this way, the representation (helper graphic element alignment 190) on the display 180 of the orientation of the slave device 170 (for example expressed in roll-pitch-yaw and/or twist-swing coordinates) can be derived from the acquired image.

Alternatively or additionally, the representation (help-alignment graphic element 190) on the display 180 may be derived by implementing computer vision and machine learning algorithms on such images. In this case, the vision system 160 preferably comprises a microscope capable of magnifying the acquired images.

For example, where the master device comprises two rigid parts pincer-hinged about a common axis, “alignment” preferably means the alignment between a longitudinal axis extending between the two rigid parts 134, 135 and through the constraint rotational 136 of the master device 110 (for example the “x” axis in FIG. 2A) and the control point 600 identifying the slave device 170.

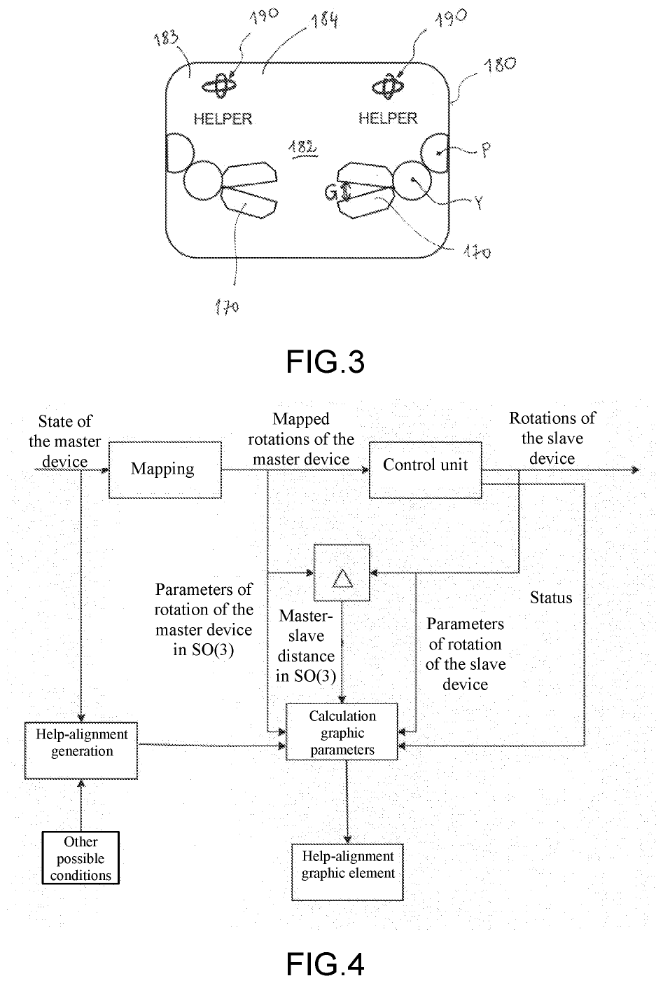

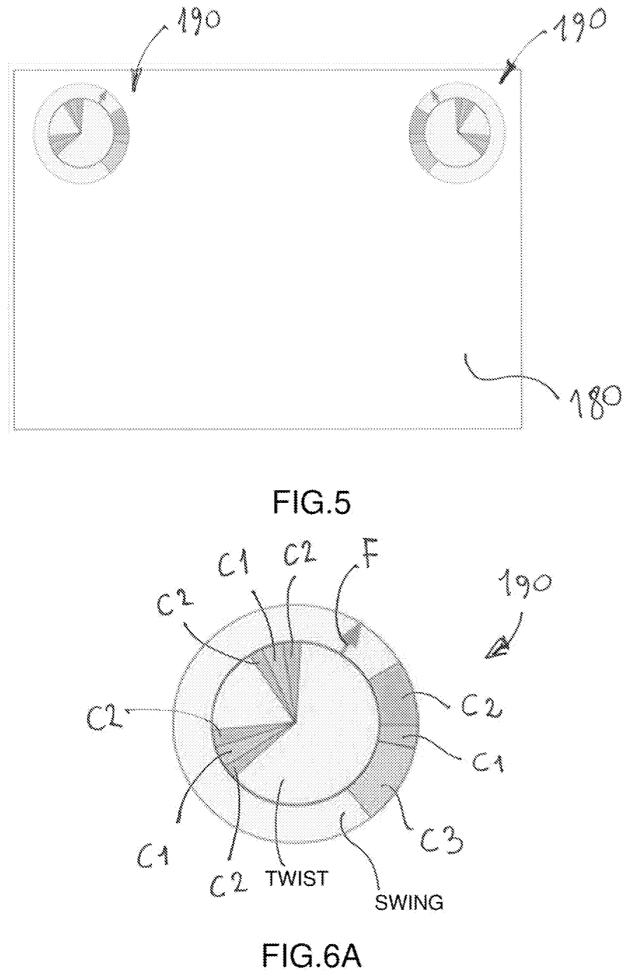

According to one aspect, the method of this disclosure provides the operation of generating with the central unit a graphical user interface on the display 180, when the central unit receives an help-alignment request signal. This graphical interface can for example be as shown in FIG. 3 and comprises an help-alignment graphic element 190, which in FIG. 3 is indicated as HELPER and which can appear like those exemplified in FIGS. 5 to 14. Thanks to an interface graphics such as the one shown in FIG. 3, the surgeon 150 can display one or more graphic elements which make it clear what is the orientation of the master device 110 relative to the corresponding slave device 170, which the surgeon sees represented on the display 180.

According to a preferred aspect, the slave device 170 is displayed in a substantially central portion 182 of the display 180, while the help-alignment graphic element 190 is displayed in a portion of the display 180 not superimposed on the central portion 182 of the display, for example in a corner 183 of the display or in another peripheral portion 184 so as not to block the view of the surgeon 150 on the slave devices 170 of the robotic system 100. Preferably, the robotic system 100 comprises at least two slave devices 170 preferably controlled by respective at least two 110 master devices.

Not necessarily a slave device 170 is displayed in a substantially central portion 182 of the display 180, and in one embodiment, the help-alignment graphic element 190 is however displayed in a portion of the display 180 that is not superimposed on the portion of the same display 180 in which the slave device 170 is represented.

According to one embodiment, no slave device 170 is shown on the display 180, and the at least one help-alignment graphic element 190 is shown on the display 180. For example, a second, further display (not shown) may be provided, in wherein the slave device 170 is displayed on said second further display, and the at least one help-alignment graphic element 190 is displayed on the first display 180.

According to one aspect of the method of this disclosure, schematically illustrated in FIG. 4, a first spatial orientation of the master device or devices and a second spatial orientation of the corresponding slave device, or slave devices, are determined with the central unit (“control unit”). The possible misalignment (i.e. the relative orientation) A between master and slave is then determined and the central unit modifies a graphic aspect of the help-alignment graphic element 190 in a manner corresponding to the relative spatial orientation of the master device with respect to the slave device, so as to provide the surgeon with visual information which makes it clear how the master is oriented with respect to the slave and allows him to achieve an alignment condition between the master device and the slave device. For example, the status or pose of the master and/or other inputs generate the alignment help request signal (“trigger”), the master is mapped to slave coordinates, and a further three inputs are processed to produce a visual representation of the feature help-alignment graph and in particular: the rotation of the mapped master (“Master rotation param”), the rotation of the slave (“Slave rotation param”) and their relative rotation in SO(3).

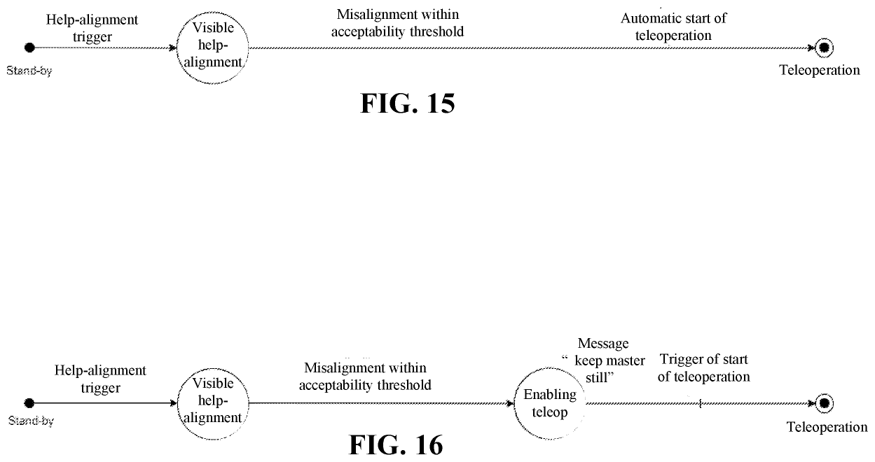

FIGS. 15 to 18 show state diagrams illustrating how to start a teleoperation after a graphical user interface has been opened according to the method of the invention on the display 180 of a robotic system 100, such as for example the illustrated robotic system in FIG. 1. Once this graphical user interface is shown on the display, various situations can occur, exemplified in the figures:

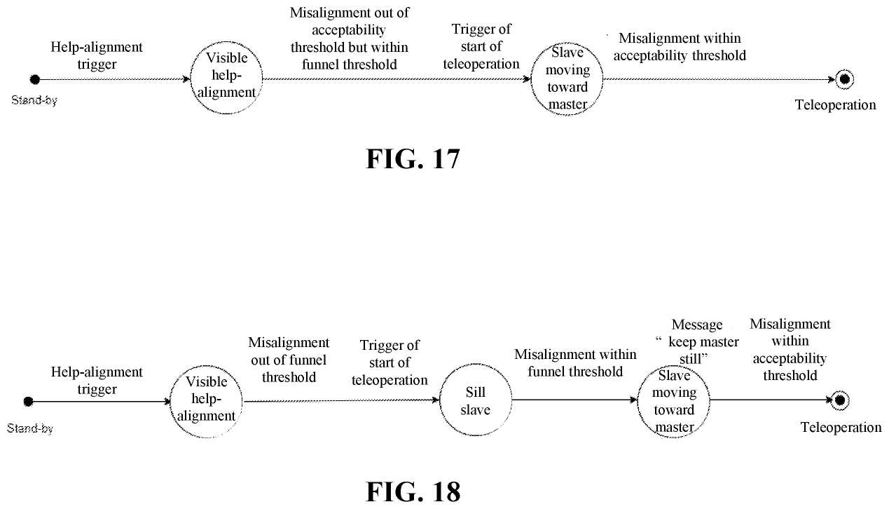

FIG. 15: after the graphical interface has been opened, if it happens that the relative
spatial orientation between the master device and the slave device is within a pre-established range, the graphical interface is closed and a teleoperation is automatically started;
FIG. 16: after the graphical interface is opened, if it happens that the relative spatial orientation between master device and slave device is within a predetermined range, the graphical interface is not closed automatically and the graphical interface informs the operator that teleoperation is ready to be enabled. The robotic system therefore waits for an enabling signal (“trigger”) of a medical or surgical teleoperation to be generated;
FIG. 17: after the graphical interface has been opened, if the relative spatial orientation between the master device and the slave device is outside an acceptable tolerance range but is included in a second maximum range (“funnel”), which includes the acceptable tolerance range, when an enable signal (“trigger”) is generated (e.g. an alignment enable signal and/or a medical or surgical teleoperation enable signal), an alignment phase begins for example with the operator holding the master device stationary and the robotic system automatically and/or autonomously moving the slave device until the relative spatial orientation between the master device and the slave device falls within the acceptable tolerance range. At that point, the graphical interface closes and teleoperation starts;
FIG. 18: after the graphical interface has been opened, if the relative spatial orientation between the master device and the slave device is outside the second maximum range (“funnel”), when an enable signal (“trigger”) for entry into medical or surgical teleoperation, starts an alignment phase, for example by keeping the slave device stationary and asking the operator, via the graphical interface, to move the master device until the relative spatial orientation between the master device and slave device is not included in the second maximum range. In this situation, the graphical interface informs the operator to keep the master device still while the slave device is moved automatically and/or autonomously until the relative spatial orientation between the master device and the slave device falls within the range of acceptable tolerance.

When this happens, the GUI closes and teleoperation starts.

According to one aspect, the graphical user interface is in any case closed when a maximum amount of time has elapsed that the graphical user interface has been open since the alignment-aid request signal was generated, and/or for example without entering teleoperation.

According to a preferred embodiment, the robotic system generates said help-alignment request signal with said central unit outside a teleoperation, and preferably in a teleoperation preparation step.

According to an embodiment, the robotic system generates said aid-alignment request signal with said central unit in a suspended teleoperation phase, for example a pause from the teleoperation envisaged between two contiguous completely slaved teleoperation phases.

According to an embodiment, the robotic system generates said help-alignment request signal with said central unit in a limited teleoperation phase in which a subset of the slave device degrees of freedom remain slaved to the master device while a second subset of degrees of freedom are blocked.

Of course, the aspects described below for the generation of the help-alignment request signal can be provided in any combination thereof. In fact, one requirement is to be able to activate an alignment procedure between the master device(s) and the slave device(s) in a simple and safe way.

According to one aspect, a requirement to the generation of the help-alignment request signal may be the detection of the master device, or of two master devices, within a predeterminable working space. Said predeterminable working space in which the master device 110 must be located for the generation of the help-alignment request signal can coincide with the entire working space 115 of the master device (defined for example by the limits of the “tracking” field) or it can be a sub-space 116 defined by the central unit. In this way, the predeterminable working space 116 in which the master device must be located in order to generate the help-alignment request signal is a subset of the master working space 115, as shown for example in FIG. 24. For example, said predeterminable workspace can exclude areas for storing the master device when not in use, such as receptacles and/or the like. For example, the predeterminable workspace may have the shape of a sphere and/or two spheres, such as for example one sphere for each master device, and/or a box. An external region may be provided and an internal region contained in the external region of the working space, wherein said predeterminable working space in which the master device must be located in order to generate the help-alignment request signal may be the external region or the outer region.

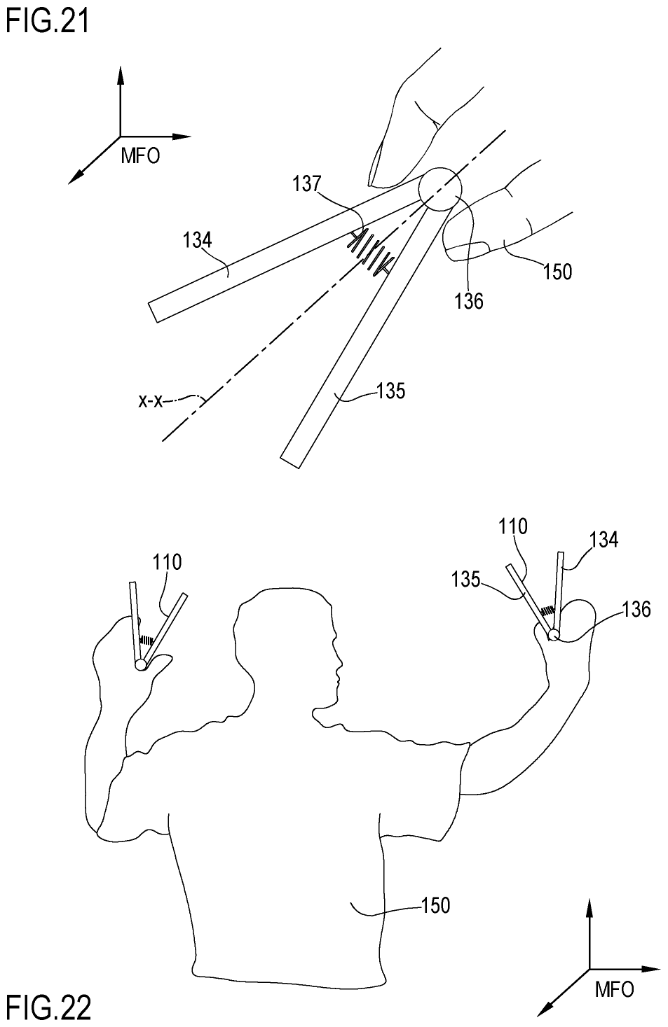

According to one aspect, the help-alignment request signal is generated by the central unit when the open/close command of the master device 110 has been activated. As shown for example in FIG. 21, a slight pressure of the open/close command (grip, G) of the master device generates the help-alignment request signal. Where two master devices are provided, such as for example a right master device intended for the surgeon's right hand and a left master device intended for the surgeon's left hand, the help-alignment request signal may be generated by the central unit when the opening/closing command of both master devices is activated simultaneously or within a time window from the first closing/opening. According to one embodiment, the help-alignment request signal is generated by the central unit when the opening/closing command of the master device has been repeatedly activated, such as for example a “double-tap”.

According to one aspect, the help-alignment request signal is generated by the central unit when the master device is detected upside down, i.e. “top-down”, as shown for example in FIG. 22. In this case, it is possible to provide further conditions such as for example the help-alignment request signal is generated when the master device 110 is detected upside down and stationary, i.e. not in free fall. For example, the help-alignment request signal can be generated in reversed master conditions and an activated open/close command. In addition, a further check to establish that the surgeon's gesture is aimed at indicating the will to start an alignment phase could be a check on the vertical position of the master device, for example to detect if the surgeon has overturned the master device by simultaneously lifting the arms, as shown schematically in FIG. 22.

According to one aspect, the aid-alignment request signal is generated by the central unit when a gaze recognition device installed in said robotic system 187 detects that the operator is looking in a predetermined direction, such as towards the screen 180 or in a certain area or part of the screen 180.

According to one aspect, the help-alignment request signal is generated by the central unit when the master device 110 is directed towards the screen 180 or display 180, according to a predefined orientation.

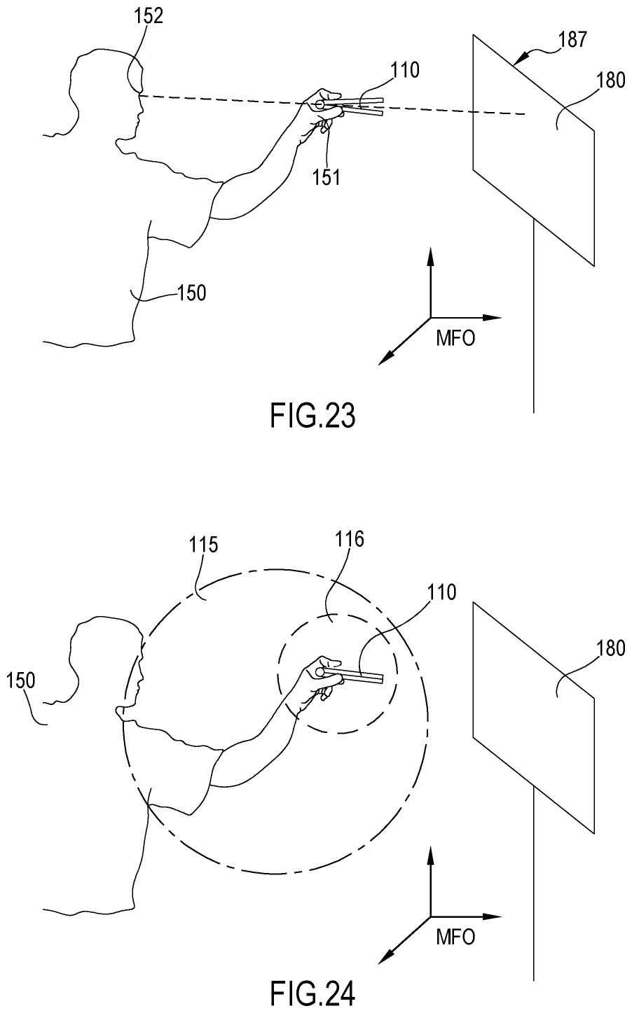

According to one aspect, the aid-alignment request signal is generated by the central unit when an alignment is detected between the hand 151 of the surgeon 150 gripping the master device 110, at least one eye 152 of the surgeon and at least a portion of the screen 180, as shown for example in FIG. 23. For example, a camera can detect such an alignment. For example, gaze sensing device 187 detects such an orientation.

According to one aspect, the method comprises the operation of generating the help-alignment request signal with the central unit when the central unit detects that a predetermined movement has been performed with the master device. In practice, the central unit determines the position of the master device(s) and, when the central unit itself recognizes a pre-established movement made by the surgeon holding the master device(s), the central unit activates the graphical user interface.

In one aspect, the central unit determines the position of the master device(s) and, when the central unit itself detects that the master device is in a predefined spatial pose or in a predefined master working space, the central unit activates the graphical user interface. By doing so, the surgeon can activate the graphical user interface by continuing holding the master device (or devices) and without taking his eyes off the display, which shows the surgical site in which the slave device (or devices) operates.

The help-alignment request signal which causes the central microprocessor unit to activate the graphical user interface can also be generated in other ways.

According to one aspect, the robotic system can be equipped with a gaze detection device 187 of the user using the robotic system, to generate the aid-alignment request signal (and therefore the opening of the graphical user interface) when the user looks in a predetermined direction.

According to another aspect, at least one or each master device 110 of the robotic system can be equipped with a specific switch 158, for example a button, to generate the help-alignment request signal when it is activated (for example pressed).

According to one aspect, each master device may be equipped with a capacitive or other sensor which, when touched by the surgeon holding the master device, generates the help-alignment request signal for the central microprocessor unit which in turn opens the graphical user interface on the display. By installing the button or capacitive sensor on the master device in a position that it can be reached with a simple movement of the fingers without changing grip, the operator can generate the aid-alignment request signal without taking his eyes off the display.

According to one aspect, if the robotic system comprises a foot switch 158 functionally connected to the central unit, when this is pressed, the help-alignment request signal can be generated and then display the graphical user interface on the display.

In one aspect, the graphical user interface can be closed when the pedal is released, whether or not alignment has been achieved between the master and slave devices.

According to one aspect, the help-alignment request signal can be generated with the central unit when the command of the master device 110 suitable for controlling the opening/closing degree of freedom G of the slave device 170.

In the event that the master device 100 comprises two rigid parts 134, 135 hinged to each other like a pincer around a common axis, for example as shown in FIG. 2A, with the two rigid parts 134, 135 kept angularly apart from the spring 137 when it is at rest and being able to be brought closer to each other only by overcoming an elastic repulsion force from the spring, the help-alignment request signal can be generated with the central unit when the two rigid parts are brought closer to each other until an angle between them is defined which is smaller than a predetermined angular threshold which takes into account the elastic influence action of the spring.

According to still another aspect, with the master devices 110 of the robotic system having two rigid gripper-hinged parts 134, 135, the alignment-aid request signal can be generated by rapidly closing the rigid gripper-like parts twice consecutively (“double-tap”). The master device 110 may comprise an elongated body provided with an opening/closing command, and this help-alignment request signal can be generated by rapidly activating the opening/closing command (“double-tap”) twice consecutively.

In one aspect, the help-alignment request signal is automatically generated by the central unit. According to an implementation option, the alignment help request signal is automatically generated by the central unit when a predeterminable condition is detected during the master-slave teleoperation. The predeterminable condition detectable during a teleoperation state may include: the slave device is near the physical limits of the slave workspace (limit of the joints, for example of the pitch P and/or yaw Y and/or roll R joints of the articulated terminal of the surgical instrument 170), and/or the pose commanded to the slave device is outside the working space of the slave device (for example outside the working space of the pitch P and/or yaw Y and/or roll R joints). The predeterminable condition detectable during a teleoperation state may include: state transition from or to a limited teleoperation state, in which for example the slave device follows the master device only in orientation and/or only in translation and/or in a subgroup degrees of freedom including orientation and/or translation.

Various examples of graphical interfaces which can be visualized on the display in the way schematized in FIG. 3, are illustrated in FIGS. 5 to 14 with reference to the case in which the robotic system comprises two master devices, intended to be gripped by the surgeon, and two corresponding slave devices operating in the surgical site, but what will be said also applies in the case where there is only one master device and one slave device and the graphical user interface therefore comprises only one graphic element. In the illustrated case where there are two master devices and two slave devices, the displayed graphical user interface will contain a first graphic aid-alignment element for the first master-slave pair and a second graphic aid-alignment element for the second pair master-slave. The central microprocessor unit determines the position of the master and slave devices and modifies the appearance of the two graphic elements so as to visually represent the current relative orientation of the respective master device from the corresponding slave device.

The graphic element that visually represents the misalignment between master and slave devices or the respective orientation of the real-time updated master and slave can be represented in various ways.

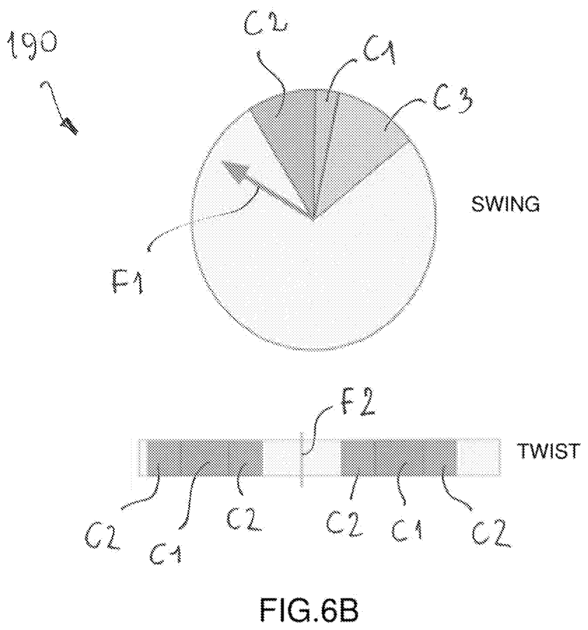

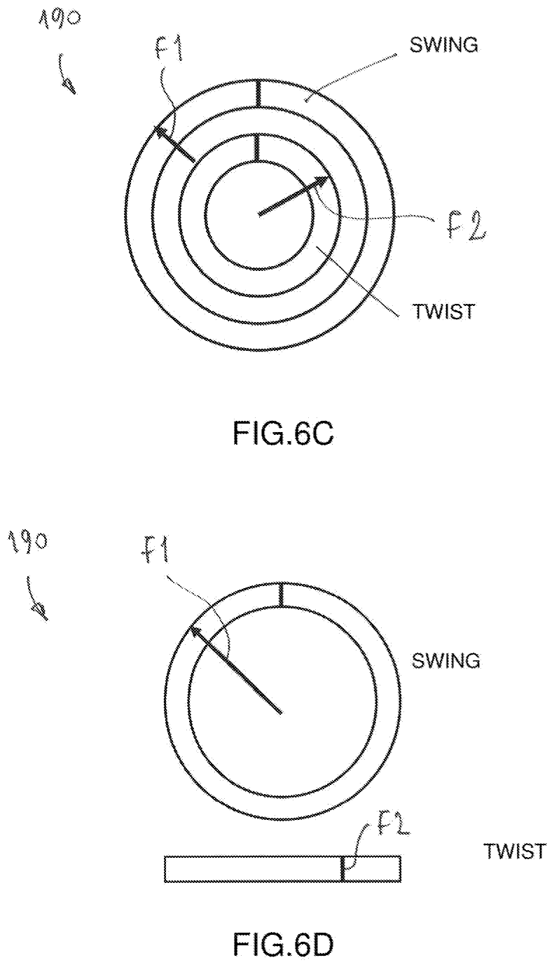

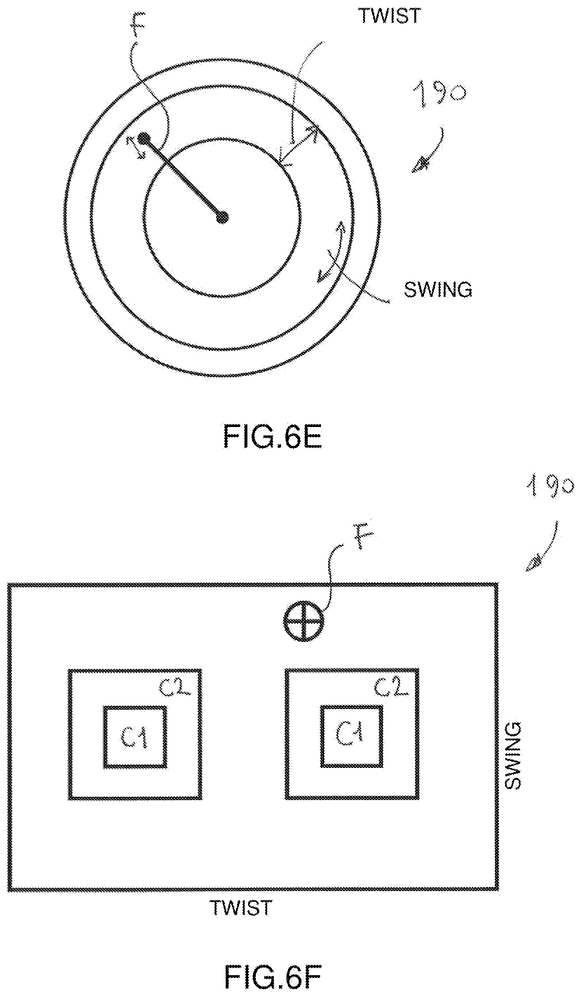

For example, as shown in FIG. 5 the two help-alignment graphic elements 190 can be located on opposite corners of the screen 180 and each can be depicted as concentric circles with circular sectors of different colors C1, C2, C3 and with at least one indicator F, F1, F2 (such as an arrow, a cursor, and the like). An help-alignment graphic element 190 of FIG. 5 is shown in greater detail in FIG. 6A, in which the inner circle represents the twist angle and the circular sector outside it represents the swing angle, and in which the arrow F represents the orientation of the master device, and in which the color C1 represents a region of misalignment lower than an acceptability threshold such as to enable teleoperation (for example 5-8 degrees), and in which the color C2 represents a region of misalignment lower than a “funnel” threshold such as to enable the movement of the slave device (for example 40-60 degrees) to align with the master device, and in which the color C3 represents a region that is external to the slave workspace. Note that, in this example, due to the 2-Fold rotational symmetry around the roll x axis of the master device 110, two possible acceptability thresholds (color C1) are shown for the twist angle (“Master Flip” mode).

For example, in FIG. 6B the twist and swing angles are represented on two distinct and separate geometric shapes, where the circle represents the swing angle and the bar represents the twist angle; in this figure, the color C1 corresponding to the acceptability threshold has been placed in the “12 o'clock” configuration; the arrows F1, F2 represent the master device 110; in this representation the colors C1, C2, C3 represent concepts similar to what is mentioned with reference to FIG. 6A.

For example, in FIG. 6C the twist and swing angles are represented on concentric annuli, similar to what was discussed with reference to FIG. 6A.

For example, in FIG. 6D the swing angle is represented on an annulus, while the twist angle is represented on a bar, similar to what was discussed with reference to FIG. 6B.

For example, in FIG. 6E the swing angle is represented by an annulus (angular representation) and the twist angle is represented as the radial extension of the same annulus; therefore, in this example, the F indicator is both able to angularly and radially move in the annulus, i.e. the annulus becomes thicker/thinner as a function of the twist angle.

For example, in FIG. 6F the twist and swing angles are represented on a Cartesian plane as two orthogonal axes and the F indicator is represented as a crosshair.

Naturally, a person skilled in the art could choose to exchange the graphical representations of the twist and swing angles, with respect to what is described with reference to these exemplary FIGS. 6 A-F.

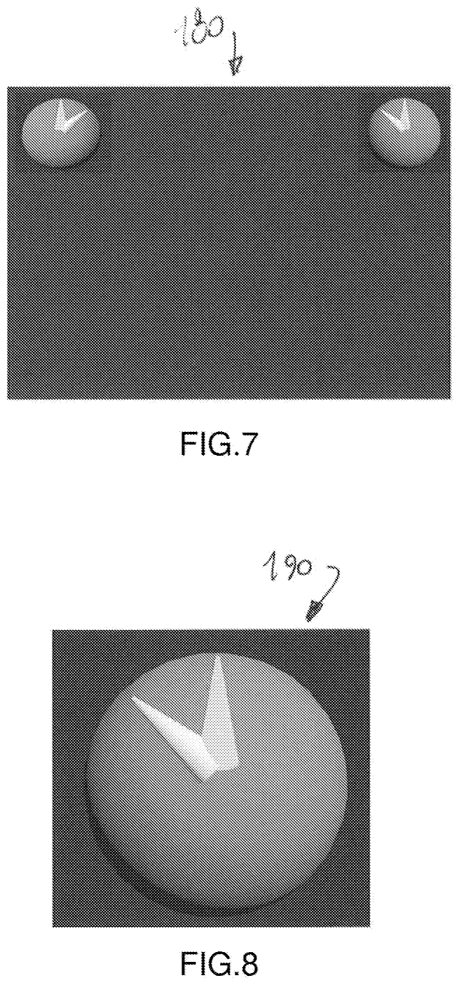

According to one aspect, each graphic element can be represented as shown in FIGS. 7 and 8 with two cones, one representing the master device and the other representing the slave device, which visually provide the information of misalignment between master and slave devices in terms of angular coordinates of a spherical reference system, in which the information on the misalignment is shown by representation of the master device and the slave device. More in detail, FIG. 8 shows a detail of the help-alignment graphic element consisting of two three-dimensional cones representing the orientation of the Master, which is updated in real time, and the relative orientation of the slave, in one embodiment they have different colors.

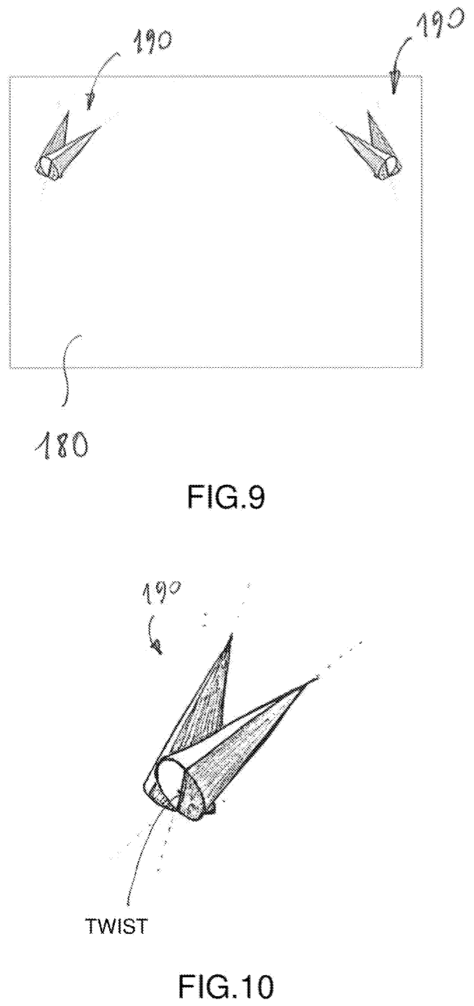

In one aspect, each graphic element may be depicted as shown in FIGS. 9 and 10 with two oblong cones. For example, the coloring of the cone may include displaying information on misalignment along the roll axis or the twist axis, i.e. along the longitudinal dimension of the master and/or slave devices. In more detail, FIG. 9 shows how to visualize on a display two right and left graphic elements respectively for alignment aid according to the present disclosure in the shape of three-dimensional cones with two-color polar identification of roll.

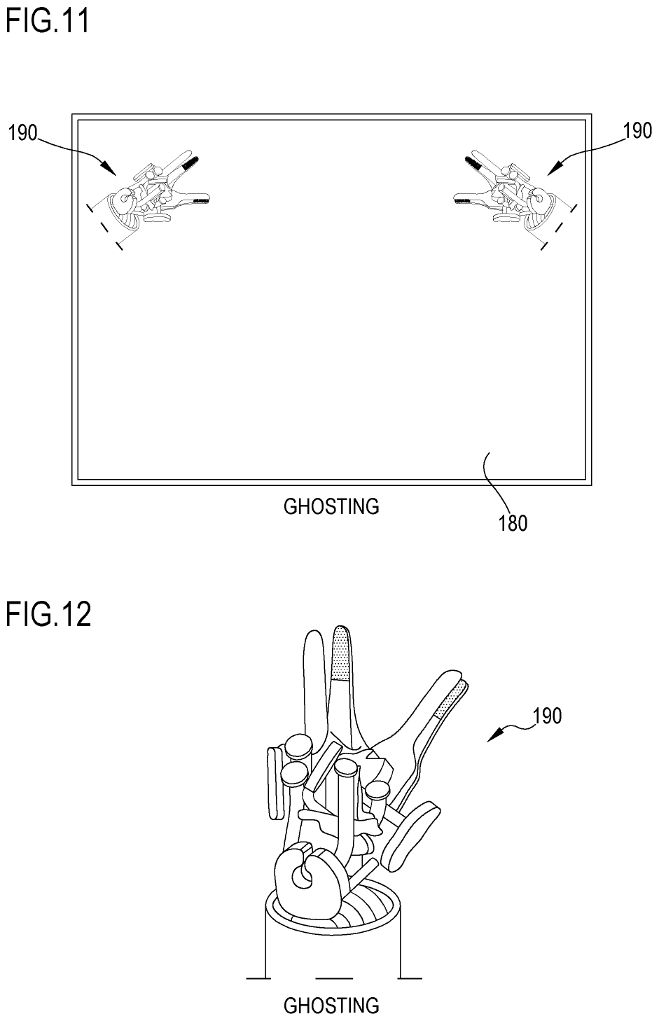

According to a preferred aspect, each graphic element is of the type illustrated in FIGS. 11 and 12 and depicts a virtual, i.e. synthetic slave device, which appears as the surgeon sees the real slave device in the central portion of the display, partially superimposed on another graphic element which depicts the corresponding master device. In the exemplified case in which the slave devices are substantially constituted by two rigid parts integral with respective sensors or markers 134, 135 constrained in a rotational joint such as a gripper to rotate around a common axis while the master device is manipulated by an operator 150, the graphic element depicting the master device and the graphic element depicting the slave device can be superimposed at the rotational joint, so that the misalignment between the rigid pincer-hinged parts of the master device he holds and of the slave device operating at the surgical site.

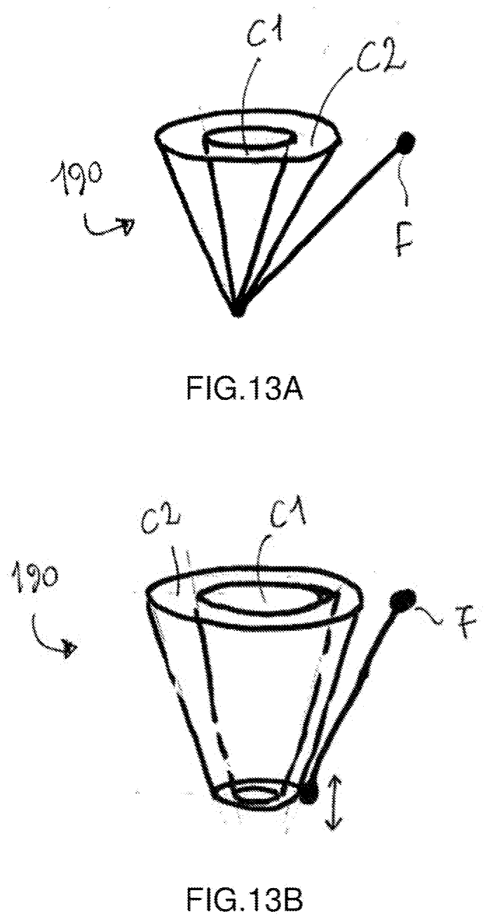

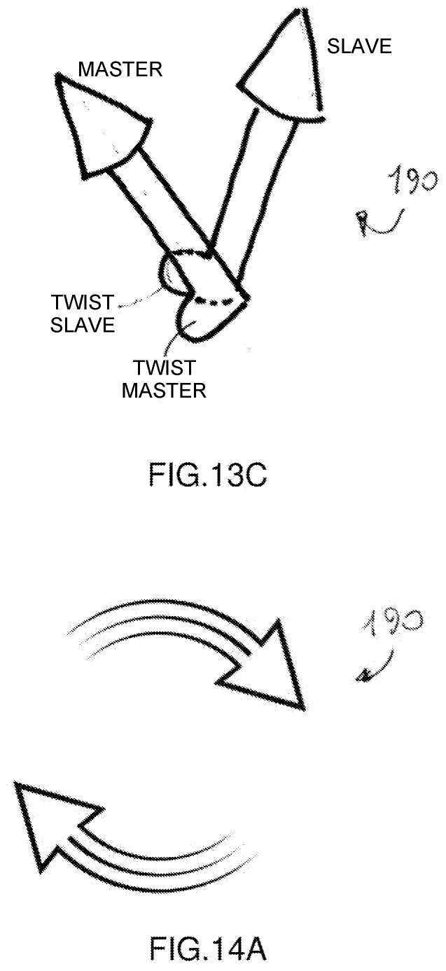

For example, in FIG. 13A, the graphic element may be represented with an arrow F which identifies the misalignment between the master and slave device and with at least one cone which visually identifies the maximum tolerable misalignment between the master and slave devices. For example the size of the arrow F may increase with misalignment. For example, two or more concentric cones colored with said colors C1, C2 may be provided to illustrate the acceptability and “funnel” thresholds (for example 5-8 degrees and 40-60 degrees respectively). Similarly, in FIG. 13B, truncated cones are shown instead of cones. For example, in FIG. 13C, two arrows are shown illustrating the master device and the slave device respectively, where the foot of the arrow indicates the twist or roll angle.

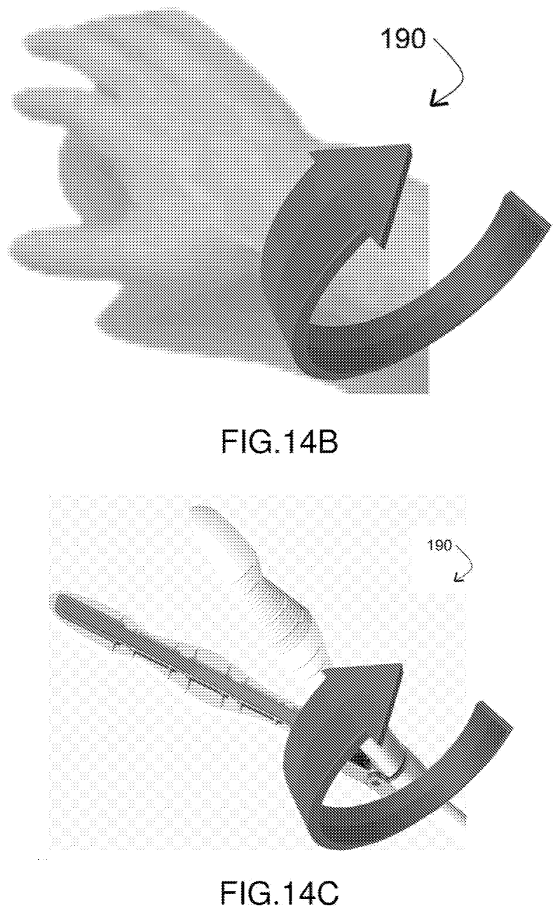

According to an aspect illustrated in FIGS. 14A-C, the graphic element can depict instructions for reaching the alignment condition, indicating to the operator how to move (rotate) the master device 110 to align it with the orientation of the slave device.

For example FIG. 14A shows two arrows; FIG. 14B shows an arrow which is superimposed on a representation of the hand 151 of the surgeon 150 to indicate a twist or roll rotation instruction; FIG. 14C shows an arrow being superimposed on a representation of the master device 110 to indicate a twist or roll instruction.

In one aspect, real-time master-slave misalignment is depicted in the form of an abstract and simplified image to increase the intuitiveness for the surgeon. In accordance with one embodiment, the misalignment is depicted in real time as a cursor running along a line or bar which may exhibit color-coded coloring indicating the misalignment, as shown for example in FIG. 6b or 6c. For example, when the misalignment is lower than a certain acceptability threshold, the cursor is positioned in correspondence with a green colored section of the line or bar, while when the misalignment is greater than this certain acceptability threshold, the cursor is positioned in correspondence with a yellow, orange and/or red colored section of the line or bar.

In accordance with one embodiment, the misalignment is depicted in real time as a hand rotating along a portion of a circle, for example an annulus. The provision of a simplified abstract representation of the relative orientation (misalignment) between master and slave allows to avoid representing redundant information making the process of alignment of the master device to the slave device intuitive.

In accordance with a preferred embodiment, the alignment-assist graphic element is not an overlay (in technical jargon: “ghosting”) on the image of the “real” slave device 170 acquired by a vision system 160. In other terms, preferably avoid making the help-align graphic element as a “ghosting” overlay. This choice makes it possible to obtain a more effective representation, which is independent of the current pose of the slave device and therefore is more understandable in any kinematic configuration of the slave device.

In fact, typically “ghosting” is based on augmented reality techniques: the system identifies the slaves in the image using “computer vision” techniques and superimposes a three-dimensional transparent model (“ghost”) on these images which shows the instrument pose mapped by the corresponding master pose. This representation has the drawback of being subject to problems of self-occlusion which makes understanding difficult, of being positioned in variable positions on the screen 180 potentially close to the edges, or of mutual occlusion between two close instruments. FIG. 12 shows an help-alignment graphic element according to the present disclosure depicting the orientation in a synthetic image of a slave device partially superimposed on an image of a respective master device to represent a relative orientation of the slave device with respect to the corresponding master device, the synthetic image in one embodiment can be a simulated or CAD reproduction of the Slave or Master device, however not associated with that potentially represented by a vision system. One of the disadvantages of ghosting in augmented reality is that the roll axis of the real surgical instrument, i.e. the longitudinal axis of the instrument, is generally prevalent, and this does not favor an adequate illustration, especially in cases where the articulated end portion (pitch, yaw, grip) of the surgical instrument is miniaturized.

Thanks to the characteristics described above, provided separately or jointly with each other in particular embodiments, it is possible to meet the above-mentioned needs obtaining the aforementioned advantages, and in particular:
where a teleoperation preparation phase is envisaged comprising an alignment phase with movement of the slave device, in which the slave is enabled to move to reach the orientation of the master device, as explained above, the provision of said at least one graphic element of aid-alignment allows to minimize the entity of the movement of the slave device during the phase of alignment with movement (FIG. 20);
the help-alignment graphic element may be generated in response to a precise command by the user;
the graphic aid-alignment element may be generated automatically, depending on the state of the robotic system;
the graphic element can appear, for example, during a teleoperation phase in which the command of the master device requires the slave device to reach the physical limits of the working space of the rotational joints of the articulated surgical instrument of the slave device;
unwanted movements of the slave device are avoided or at least reduced to a minimum;
a simple and robust solution is provided which allows a clear and intuitive representation for any pose of the slave device, i.e. spatial configuration of the joints of the surgical instrument of the slave device;
in particular, by avoiding the creation of said at least one help-alignment graphic element in the form of “ghosting” on the real slave acquired by a vision system, a clear and intuitive and thus effective visualization is permitted, even when the kinematic configuration of the joints of the slave device is unfavourable, i.e. for example such that the control point 600 is hidden by the body of the surgical instrument itself or by the body of a further second surgical instrument.

The various embodiments of the robotic system control method of this disclosure may be implemented by making a computer program, which is loaded into an internal memory of the central microprocessor unit of the robotic system and which, when launched, causes the central microprocessor unit to carry out the operations of the control method described above.

LIST OF REFERENCE NUMBERS

| | |
|---|---|
| 100 | Robotic system for medical or surgical teleoperation |
| 110 | Master device |
| 115 | Master workspace |
| 116 | Predeterminable subspace of the master workspace |
| 134 | First rigid part of the master device |
| 135 | Second rigid part of the master device |
| 136 | Articulation of the master device |
| 137 | Elastic element of the master device for the opening/closing |
| | command |
| 142 | First flagship link of the slave device |
| 143 | Second flagship link of the slave device |
| 150 | operator or surgeon |
| 151 | Surgeon's hand |
| 152 | Surgeon's eye |
| 158 | Switch |
| 160 | Vision system, for example camera |
| 170 | Slave device or slave device surgical instrument |
| 180 | Display or screen |
| 182 | Central portion of the screen |
| 184 | Peripheral portion of the screen |
| 187 | Eye tracking device |
| 190 | Help-alignment graphic element |
| 600 | Checkpoint |
| MF | Master local reference system |
| SF | Slave local reference system |
| MFO | Master workspace reference system |
| SFO | Slave workspace reference system |
| R | Degree of freedom of roll |
| P | Pitch degree of freedom |
| Y | Degree of freedom of yaw |
| G | Degree of freedom of opening/closing, or grip or seize |
| X-X | Longitudinal axis of the master device |
| C1, C2, C3 | Colors |
| F | Indicator |

The invention claimed is:

1. A control method of a robotic system for medical or surgical teleoperation, wherein said robotic system comprises at least one master device, which can be handled and is adapted to be moved by an operator, at least one slave device configured to be moved by an actuator and to be controlled by the master device, a central microprocessor unit configured to control said slave device, and a display,
said method comprises performing the following operations:
determining with said central unit a first spatial orientation of said master device and a second spatial orientation of said slave device;
when said central unit receives a help-alignment request signal, opening with said central unit a graphic user interface on said display, said graphic user interface comprising at least one help-alignment graphic element displayed in a portion of said display, visually representing information on a relative spatial orientation of the master device updated in real time with respect to the slave device and/or instructions to allow said operator to reach an alignment condition between the master device and the slave device;
updating said help-alignment graphic element shown on the display in a manner corresponding to the relative spatial orientation of the master device with respect to the slave device;
closing, with said central unit, said graphic user interface when at least one of the following conditions is met:
said relative spatial orientation is within a predetermined interval;
an enabling signal for a medical or surgical teleoperation has been generated,
a maximum time has elapsed wherein said graphical user interface has remained open since said help-alignment request signal was generated.

2. The method according to claim 1, wherein the master device is of the unactuated type.

3. The method according to claim 1, wherein said display is configured to display at least in its central portion a surgical site in which the slave device operates,
the method including the following steps:
displaying said surgical site and said slave device in said central portion of the display;
displaying said help-alignment graphic element in a peripheral portion of said display, not superimposed on said central portion of the display, which shows said slave device.

4. The method according to claim 1, comprising the operation of displaying the at least one help-alignment graphic element on said display, avoiding viewing the surgical site; or wherein said robotic system includes at least a first display and a second display, the method comprising the operations of viewing the surgical site on the first display and displaying the at least one help-alignment graphic element on the second display.

5. The method according to claim 1, comprising the operation of generating said help-alignment request signal with said central unit out of a teleoperation.

6. The method according to claim 1, comprising the operation of generating said help-alignment request signal with said central unit in one of the following conditions, or their combination:
when the closing/opening command of the master device has been activated;
when the opening/closing command of the master device has been repeatedly activated;
when the master device has been overturned by a hand gesture in a defined time interval;
when a gaze detection device installed in said robotic system detects that the operator is looking in a predetermined direction;
when the master device is subjected to a predetermined hand gesture, in a predefined time interval.

7. The method according to claim 1, comprising the operation of generating said help-alignment request signal with said central unit in one of the following conditions, or their combination:
when said central unit detects that the master device is in a predetermined workspace;
when said central unit detects that a predetermined movement has been performed with said master device;
when said central unit detects that said master device is in a predefined spatial position;
when said central unit detects that a hand-activated switch of the robotic system has been pressed, said hand-activated switch being installed on a handle of said master device and being connected to said central unit;
when said central unit detects that a foot switch of the robotic system has been pressed, said foot switch being connected to said central unit.

8. The method according to claim 1, comprising the operation of generating an help-alignment graphic element to:
visually represent the relative spatial orientation between said master device and said slave device.

9. The method according to claim 1, comprising the operation of generating an help-alignment graphic element so as to:
visually represent said first spatial orientation of the master device and said second spatial orientation of the slave device.

10. The method according to claim 1, comprising the operation of generating an help-alignment graphic element so as to:
visually represent information, such as a graphic indicator, to move the master device to achieve alignment with said second spatial orientation of the slave device.

11. The method according to claim 1, wherein when said relative spatial orientation is within a predetermined interval, an enabling signal for a medical or surgical teleoperation is generated.

12. The method according to claim 1, wherein when said relative spatial orientation is within a predetermined range, a slave device motion enable signal is generated to align with said master device orientation;
and wherein when said relative spatial orientation is within a narrow range, contained in the predetermined range, an enabling signal for a medical or surgical teleoperation is generated.

13. The method according to claim 1, wherein said enabling signal of a medical or surgical teleoperation is generated by a request from the operator.

14. The method according to claim 1, wherein said signal for enabling a medical or surgical teleoperation is automatically generated when said relative spatial orientation is within a predetermined range.

15. A computer program installable in an internal memory of a microprocessor unit, comprising a software code which, when run, causes said microprocessor unit to perform the operations of the method according to claim 1.

16. A robotic system for medical or surgical teleoperation, comprising at least:
a master device that can be handled by an operator,
a slave device comprising at least one surgical instrument configured to be moved by an actuator and to be controlled by the master device,
a central microprocessor unit configured to control said slave device,
a display;
wherein the computer program according to claim 15 is installed in said central microprocessor unit.

17. The robotic system according to claim 16, further comprising a foot switch functionally connected to said central unit, said foot switch being configured to generate the help-alignment request signal when pressed.

18. The robotic system according to claim 16, wherein said master device is of the unactuated type.

19. The robotic system according to claim 16, comprising two displays, wherein one display is configured to display said help-alignment graphic element and the other display is configured to view the surgical site.
